(19) United States
(12) Patent Application Publication
(10) Pub. No.: US 2023/0395209 A1
(43) Pub. Date: Dec. 7, 2023

(54) DEVELOPMENT AND USE OF FEATURE MAPS FROM CLINICAL DATA USING INFERENCE AND MACHINE LEARNING APPROACHES

(71) Applicant: Verantos, Inc., Menlo Park, CA (US)

(72) Inventors: Daniel Riskin, Menlo Park, CA (US); Anand Shroff, San Carlos, CA (US)

(21) Appl. No.: 17/830,127

(22) Filed: Jun. 1, 2022

(51) Int. Cl. G16H 10/60; G16H 10/20

(52) U.S. Cl. CPC G16H 10/60; G16H 10/20

(57) ABSTRACT

Systems and methods are described for using inference algorithms and machine learning techniques to generate a clinical knowledge set. The present technology also provides systems and methods for generating feature maps comprised of patient-specific extracted and consolidated clinical features for a patient. The present technology also provides systems and methods for building a patient feature map by applying inference algorithms and a machine-learned clinical knowledge set. Such generated patient feature maps are useful for improving the care of patients.

### BACKGROUND

With newly available electronic health data and a rapid increase in processing power, data-driven personalized medicine is just now becoming possible. However, advances to improve health care are inherently limited by data quality. Care delivery improvements and payment reform rely on high quality data. Poor data quality can result in dangerous care.

In terms of data quality, the patient phenotype is critical. Phenotype is patient characteristics, which may include demographics, problems, symptoms, signs, findings, procedures, medications, laboratory studies, radiology results, and other personal and clinical characteristics. Real-world evidence, disease management, population health, and other programs to improve care rely on understanding characteristics of individual patients to select those patients within a population that should receive specific treatment or support for a specific problem.

Knowing patient characteristics within a population is a surprisingly difficult challenge. For primary use, as in care delivery, and for secondary use, as in research studies, the phenotype must be computable, or machine-readable. But, a computable phenotype requires either that the information was input in coded fashion or that a machine is able to interpret uncoded content and make it machine-readable. Since 80% of patient information is unstructured, or uncoded, this is not a trivial challenge.

To date, the large majority of computer-based systems use only structured or coded data. Within an electronic health record (EHR), these are called problem lists, procedure lists, medication lists, and other lists. Structured data represents approximately 20% of patient information within the EHR. But, these lists, particularly the problem list, are time-consuming to maintain. In a 15 minute patient encounter, the physician may have three minutes to write a narrative note for continuity of care and may dedicate 30 seconds or no time at all to updating the problem list. Peer-reviewed literature shows that problem list accuracy levels often fall below 50% in medical conditions such as heart failure and cancer. Symptoms, signs, and findings, which are rarely entered as structured data, are typically represented in lists with less than 10% accuracy. In healthcare, the patient phenotype is typically not computable due to workflow limitations and inaccuracy in the system. The data exist in the unstructured record, but are inaccessible.

Poor computable data puts current efforts in care delivery and future efforts in precision medicine in peril. There is need, therefore, to develop technology to proactively enhance the computable phenotype from source data, moving beyond complete reliance on current manual approaches.

### SUMMARY

The present technology provides systems and methods for generating feature maps comprised of patient-specific extracted and consolidated clinical features. In related embodiments, the present technology also provides systems and methods for extracting and analyzing clinical features from medical information using inference algorithms and a machine-learned clinical knowledge set, which is useful for generating the feature maps. Such generated feature maps are useful for improving the care of individual patients as seen in disease management and population health. When compiled to compare against inclusion and exclusion criteria to obtain a cohort of patients, the feature maps also build the foundation for value-based healthcare and precision-medicine research.

In one embodiment, the present disclosure provides a method for generating a clinical knowledge set, comprising identifying, from one or more medical information sources, groups of clinical features that are present together in at least one of the sources; for each group of features, using a machine learning technique to determine likelihood of relationship; and generating a clinical knowledge set with the identified groups of related features that meet a minimum threshold of relationship likelihood.

In some embodiments, the medical information sources comprise unstructured data from an electronic health record. In some embodiments, the medical information sources comprise medical literature.

In some embodiments, the likelihood of relationship is determined at least in part based on a ratio of actual frequency of co-occurrence to the likelihood of the group co-occurring by random chance. In some embodiments, each group of features is a pair of features.

In some embodiments, the features of at least one of the groups of features have a directional relationship. In some embodiments, the actual frequency of co-occurrence is determined from narrative notes of electronic health records. In some embodiments, the actual frequency of co-occurrence is determined from medical literature.

In some embodiments, the likelihood of relationship is determined at least in part based on an industry standard terminology. In some embodiments, the likelihood of relationship is determined at least in part based on a token distance of the group members and the comparison thereof with the average token distance if the group members were present together by random chance.

Another embodiment of the present disclosure provides a method for generating a feature map for a patient, comprising extracting, from a patient's medical information, a list of clinical features; identifying, for each feature in the list, associated features within the list, wherein the association is according to a clinical knowledge set; and determining features that have a threshold level of associated features within the list, thereby generating a feature map for the patient that includes clinically relevant features.

In some embodiments, the patient's medical information comprises unstructured data from an electronic health record. In some embodiments, features that are active are incorporated into the patient map. In some embodiments, features that are real are incorporated into the patient map. In some embodiments, the method further comprising maintaining, from a group of associated features within the feature map, clinically meaningful features.

In some embodiments, between two similar features, the more granular feature is incorporated into the patient map. In some embodiments, between two similar features, where a disease explains a clinical finding such as a symptom, sign, or exam finding, the disease is incorporated into the patient map but the finding is not.

In yet another embodiment, a method is provided for interpreting clinical results, comprising generating a phenotype, for each of a plurality of patients, from a feature map extracted from the patient's electronic health record (EHR); comparing the phenotype against inclusion and exclusion criteria of a study to obtain a study cohort; and identifying exposures and outcomes within the feature map to interpret clinical results for a cohort of patients.

In some embodiments, the method further validates the phenotype against a subset of the feature map that is manually curated.

### BRIEF DESCRIPTION OF THE DRAWINGS

FIG. 1 illustrates an example process of generating a clinical knowledge set.

FIG. 2 illustrates an example process of obtaining a feature map for a patient.

FIG. 3 illustrates an example process of using a phenotype based on feature maps.

FIG. 4 is a schematic illustrating the computing components that may be used to implement various features of the embodiments described in the present disclosure.

### DETAILED DESCRIPTION

Rooted in computer technology, the present disclosure provides an improved approach for extracting and analyzing clinical features from medical information using inference algorithms and a machine-learned clinical knowledge set. To enable applications, a feature map can be generated for each patient applying the clinical knowledge set to patient-specific extracted and consolidated clinical features. The feature map represents a computable phenotype for that patient. Feature maps for a group of patients can then be compiled to compare against inclusion and exclusion criteria to obtain a cohort of patients. This cohort may be applied to primary data use for care delivery or to secondary data use for value-based healthcare, research, and innovation. The patient cohort, which may be linked with exposures and outcomes, provides a powerful tool for interpreting clinical data. This new approach does not rely on manually generated lists which are prone to errors. Because it enables high accuracy clinical-grade or research-grade data, it opens a new pathway to personalized patient care, population health management, research, and value-based payment models.

### I. Clinical Knowledge Sets

A clinical knowledge set can be built from medical information sources, such as electronic health records and medical literature, to support natural language processing (NLP) and inference. Once built, the clinical knowledge set can be stored in a database and be queried during other processes of the present technology.

The clinical knowledge set can be helpful in defining the existence or even strength of relationships between clinical concepts. This is more than the conventional concept relationship based on natural language meanings. Take a conventional concept relationship as an example. The concept “diabetes with peripheral neuropathy” defines a patient having diabetes and a sequela of diabetes, peripheral nerve disease. Both “diabetes” and “peripheral neuropathy” are standard concepts defined in conventional concept relationship databases, such as SNOMED CT (Systematized Nomenclature of Medicine-Clinical Terms), which provide hierarchical relationships between these concepts.

There are a few deficiencies with such conventional hierarchical relationship databases. First, manually curated knowledge databases are limited by expensive clinician time. For example, SNOMED maintains a handful of relationships per concept. But, most concept relationships are not known if there are billions of concept relationships in healthcare, it is impossible to manually curate a database of these.

Second, the conventional hierarchical relationships provide no information about the strength of a relationship. It simply notes whether a relationship exists. For example, if a doctor writes “nausea and vomiting after eating spoiled meat,” the nausea and vomiting are likely due to food poisoning (probability˜=0.99). But, if the doctor writes, “History of diabetes and migraine headaches, now presenting with peripheral numbness.” Should one link headaches to the peripheral numbness (probability˜=0.05)? What about diabetes and peripheral numbness (probability˜=0.85)?

Third, multiple associations (e.g., many-to-one rules) and chronological relationships are generally not available in a conventional hierarchical relationship database. Moreover, the time course of associated concepts is also not provided in the conventional hierarchical relationship databases.

Fourth, in conventional hierarchical relationship databases, there is little directionality. For example, chest pain may almost always occur with heart attack, but heart attack does not always occur when there is chest pain. Directional understanding of likelihood of relationship becomes important when distinguishing likelihood that concepts are related.

1. Groups of associated clinical features and association evaluation

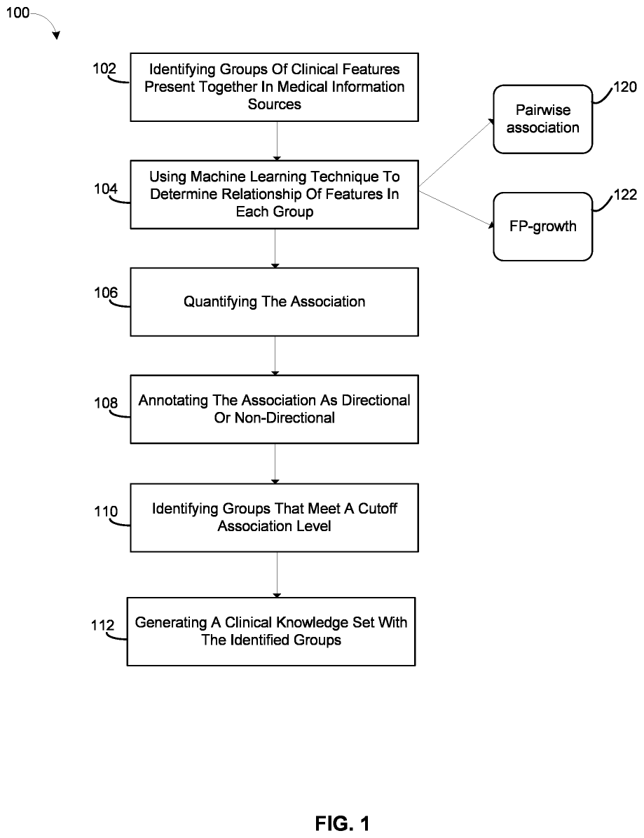

The clinical knowledge set can include groups of potentially associated clinical concepts, which can be identified from medical information sources (e.g., step 102 of FIG. 1). Some of the groups may include only two concepts (“concept pairs,” e.g., the pair of fever and infection) while others can include multiple concepts (“concept groups,” e.g., the group that includes myocardial infarction, EKG, and troponin). The initial groups of potentially associated clinical concepts can be built with relatively relaxed requirements from medical information sources, which can then be evaluated and filtered.

Medical information sources, without limitation, can include medical records (e.g., electronic health records) and medical literature. Both structured and unstructured data from the medical records may be used. Medical literature can include peer-reviewed medical journal articles, books, online publications, conference abstracts, and government reports, without limitation. In some scenarios, multiple records or documents may be concatenated into a single document. This can increase co-occurrence frequency, which is described in more detail below. In particular, the concatenation can be carried out for related records, such as the medical records for the same patient at different times.

Concerning generation of the initial concept groups of potentially associated concepts, for instance, all clinical concepts that appear in a single sentence can be drawn into an initial group. In another example, a clinical concept that appears most frequently in a medical record or literature can be first chosen as a seed for a group, and then all concepts that appear within a certain distance from the seed can also be included in the group (step 102).

In some embodiments, to reduce or avoid inappropriate associations, ambiguous terms can be excluded. For example, if myocardial infarction and chest pain co-occur, this will increase calculated lift. But, if CP and myocardial infarction co-occur, this may be ignored since CP may be either chest pain or cerebral palsy. As another example, if myocardial infarction and EKG co-occur, this will be used since myocardial infarction and EKG each resolve into only one canonical concept.

The initial groups can then be evaluated with respect to the association between the concepts in the groups, using machine learning techniques (e.g., step 104 of FIG. 1). Concept association can be considered frequent itemset mining, for which pairwise association, a priori and FP (frequent pattern)-Growth are useful evaluation methods.

An example form of concept association is pairwise co-occurrence counts (e.g., option 120 in FIG. 1). In this method, the number of times two concepts appear in the same document (e.g., a medical record, or a medical article). This is a straightforward and effective evaluation of the relationship between concepts.

Another example evaluation, and also group generation and growing, method is FP-Growth (e.g., option 122 in FIG. 1). FP-Growth defines a tree structure that is subsequently reduced to find frequent itemsets. It does not need to find all itemsets of order n in order to find those of order n+1. In this way, it is much more efficient for finding many-to-one relationships.

Specific measurement parameters can also be used to quantify the association between concepts (e.g., step 106 in FIG. 1). One such parameter, referred to herein as “lift,” is a ratio of actual co-occurrence to expected co-occurrence. For example, myocardial infarction and chest pain may occur frequently within the same encounter or longitudinal record. If actual co-occurrence is 10% and expected co-occurrence (by random chance) is 0.1%, then the lift is 100. This is a signal suggesting a real association. Expected co-occurrence may be calculated based on actual frequency of occurrence of each concept within the dataset, by prevalence of concepts within the medical literature, or by another means.

Another parameter is average token distance, which measures the average distance between pair occurrences. For example, cough may co-occur frequently with hypertension, but they may be far apart in the longitudinal record. They may be on average 20 words apart. On the other hand, myocardial infarction and chest pay may co-occur at an average of 5 words apart, typically because they are mentioned in the same sentence. This is a signal suggesting a real association. As alternatives to average token distance, median token distance, minimum token distance and maximum token distance may also be used, without limitation. Tokens may be terms that resolve to clinical concepts, may be words, or may be another measure of text distance.

Additional factors may also be considered when filtering the groups. For instance, if a pair or group of concepts can map to associated concepts in a conventional concept relationship database, such as SNOMED CT, then such a pair or group can be considered to have strong or confirmed association between the group members. An example relationship in SNOMED CT is being a child or a grandchild of the associated concept.

In some embodiments, an association value may be used to reflect the strength of association. In some embodiments, the association value may be between 0 and 1 where 1 is the strongest association. For example, due to co-occurrence and relative distance between cough and pneumonia, these may have an association value of 0.7 whereas the association value of a cough-diabetes pair may be 0.2 because these co-occur less often and less closely and are less likely to be related.

In some embodiments, each association can be further annotated as directional or non-directional (e.g., step 108 in FIG. 1), which can be reflected from the association value calculated. For example, chest pain in the presence of myocardial infarction may be far more likely than myocardial infarction in the presence of chest pain since most patients with a heart attack have chest pain but most patients with chest pain do not have a heart attack.

### 2. Thresholding

These parameters, e.g., lift and token distance, can be used to filter the groups (e.g., step 110 in FIG. 1). The filtration can be done with predetermined threshold values, or threshold values determined on the fly, e.g., to limit the number of groups. For instance, a threshold lift value may be 5, and a maximum average token distance threshold may be 20 tokens.

Alternatively, some or all of these parameters can be pooled or summarized to provide a unified association value. For instance, the association value can be defined as:

$$association coefficient = \frac{lift}{average token distance} \times ( S N O M E D relationship + 0.1 ) .$$

The score for SNOMED relationship may be arbitrary, but it can also be standardized. For instance, when all the concepts in a group map to concepts in SNOMED as child/grandchild, then the SNOMED relationship score can be considered 1.

The threshold association value can be determined based on clinical input. For instance, an association value threshold above which associations are considered acceptable can be defined to achieve an approximately 80% accuracy.

Upon such thresholding to remove groups having weak or lacking associations, the clinical knowledge set can be considered generated (e.g., step 112 in FIG. 1).

### 3. Accuracy Assessment

After the clinical knowledge set is generated, it can be assessed for accuracy. It is known that systemic collocation errors can influence numbers. For example, in a newborn screening template, there may be checkboxes for cerebral palsy, spina bifida, and fetal alcohol syndrome. These are unrelated concepts, but can look related based on NLP not recognizing the text as a template. Thus, a clinician may look through associations to find strong correlations for incorrect associations, such as a concept association value of 0.75 for cerebral palsy-spina bifida. A deep dive into the data will usually find a template or another source of systemic error. If several of these are found, it may require all data to be rerun. Thus, it is helpful to run a small number of documents, look for systemic error of associations, run a slightly larger number of documents, look for systemic error of associations, etc.

The clinical knowledge set is a learned knowledge dataset and thus may never be perfect. Once it is good enough, as deemed by the clinical informaticist, manual review may result in a manually selected concept association value threshold. For example, if 80% of the associations above an association value threshold of 0.43 are correct, then all associations above this association value threshold may be considered real and be used as the final association database.

### II. Inference to Generate Feature Maps

The clinical knowledge set can be used to help generate a feature map for a patient. As used herein, a “feature map” of a patient refers to an enriched dataset based on criteria relevant to the use case. For example, a real-world evidence study may require a feature map that includes active, real, meaningful, and unique clinical features that are extracted from the patient's medical information, and are applicable to clinical studies.

Generating a feature map from a longitudinal record, which typically includes hundreds or thousands of clinical features, however, can be a great challenge. Not all of these clinical features should be included in the feature map since not all are relevant to the use case. Typically, out of the thousands of features that can be extracted from a longitudinal record, less than a hundred should be included in a feature map.

1. Extraction of Clinical Features from Medical Record

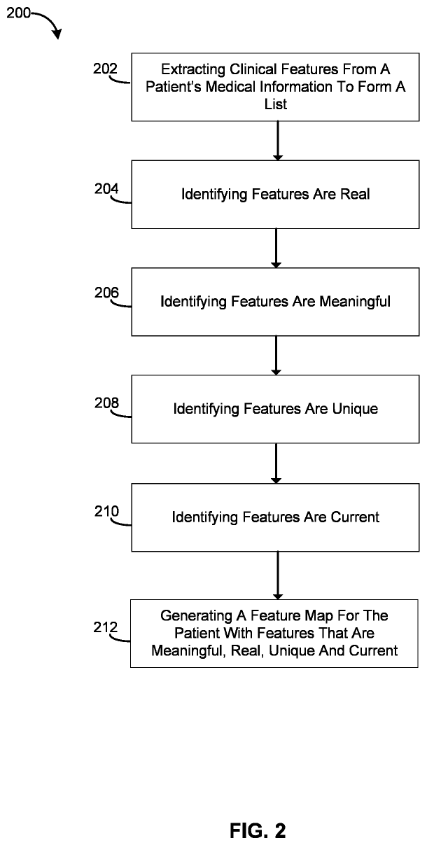

From the medical information of a patient, which may be information from electronic health records, patient reported outcomes, sensors, or other medical content, clinical features may be extracted (e.g., step 202 in FIG. 2). It is readily appreciated that the medical information typically includes both structured data and unstructured data. Structured data includes problems, medication, lab, and other coded lists. Unstructured data typically constitutes the majority of electronic health record content, including physician notes, study reports, and other provider notes such as those from nurses and social workers.

Clinical feature extraction is a specialized text extraction which is a process of extracting words and phrases from natural language narrative text that may be relevant to the health or medication condition of a patient. Simple text matching can be done with text matching software against known words and phrases in a suitable vocabulary, such as the clinical knowledge set. A more robust approach, natural language processing, may recognize subject or negations as in “a brother with cancer” or “no hypertension.” A more robust approach combines natural language processing with inference as in “Patient with high glucose, uncontrolled DM,” where DM can be recognized as diabetes mellitus based on inference from nearby mention of high glucose. A more robust approach combines natural language processing, inference, and pattern recognition as in “Patient with MA. He describes worsening headache and light sensitivity,” where the pattern of headache, light sensitivity, and MA is far more likely to be migraine with aura than mass.

Extracted features may undergo natural language processing, in some scenarios. Non-limiting examples of cleanup and tagging during natural language processing include removal of special characters, tokenization, sentence splitter, part-of-speech tagger (e.g., tags tokens with part of speech tags such as adjectives, proper nouns), named entity recognition (which matches tokens against an internal map of entities); and negation and subject tagging.

The extracted features can go through one or more tests to identify concepts relevant to the application. As examples, tests may include active, real, meaningful, and/or unique. This may be used to determine the relevant feature map for the patient. As an example of a “active” test, the feature “heart attack” extracted from “pt with h/o heart attack” refers to a past condition and thus is not active. As an example of a “real” test, “pt ruled out for heart attack” reflects negation and “brother with heart attack at age 40” reflects a different subject and thus both are not real in relation to the studied patient. As an example of a “meaningful” test, “heart attack” from “pt had a small heart attack 20 years ago” with no other mention of heart-related problems in the patient's records reflect a condition that is not meaningful for that patient. As an example of a “meaningful” test, the feature “chest pain” extracted from “pt has chest pain and heart attack” is not meaningful since “chest pain” is a symptom that can be explained by the disease “heart attack.” As an example of the “unique” test, if both “anterior heart attack” and “heart attack” are extracted features, only the former needs to be kept as it is a more granular of the latter, which does not provide additional meaningful information and can be removed.

### 2. Inference of Whether a Concept is Active

In some embodiments, each concept is checked as to whether it is active (e.g., step 210 in FIG. 2). Whether a concept is active may depend on context, chronicity, and other factors. For instance, if heart attack is discussed in the context of history, e.g., “pt with h/o heart attack,” then the heart attack is not active.

A concept in history of present illness may be more likely to be active than one in past medical history. For example, in “History of present illness: 73 year old man with leg fracture,” leg fracture can be assumed to be active. Alternatively, in “Past medical history: leg fracture, myocardial infarction, etc.” leg fracture may be assumed to be an event in the past.

A concept that is chronic can be assumed to be active. For example, in “Past medical history: Diabetes mellitus”, diabetes mellitus may be assumed to be active because it is a chronic condition.

### 3. Inference of Whether a Concept is Real

In one embodiment, a test is applied to determine whether a feature is real (e.g., step 204 in FIG. 2). For instance, when a record of “Evaluated patient for possible heart attack” is present in the electronic health record of a patient, it does not necessarily mean that “heart attack” is real. A real feature is likely to be supported by more associated features in the medical information than a non-real feature, which is more likely to be used alone. Another example where a feature is not real is when the feature refers to a different person (e.g., “brother with heart attack at age 40”) or is referred to in a negative sense (e.g., “pt ruled out for heart attack”).

In some embodiments, therefore, for each feature in the list of extracted clinical features, its associated features in the list are identified. This can be done with the clinical knowledge set that has been generated from the previous section. In some embodiments, a count of associated features in the list is calculated for each feature. A cutoff value of the count is then used to remove features that do not meet the cutoff requirement. The remaining features are considered to have passed the “real feature” test.

The cutoff value may be predetermined from some training exercises. Examples are 2, 3, 5, 7, or 10, without limitation. In some embodiments, the cutoff value is determined based on the length/size of the medical information available for the patient. Longer health records may allow higher cutoff values, for instance. In some embodiments, the cutoff value is determined individually for each patient. For examples, all features from the list can be ranked by the count of associated features, and the cutoff value may be determined to retain the top 1%, 2%, 5% or 10% features.

In some embodiments, a concept association value has been calculated for each association in the clinical knowledge set. Accordingly, alternative to in addition to the count, a sum of concept association values of all the associations between a feature and associated features in the list can be calculated. Like the cutoff value for the counts, a cutoff value for the sum of concept association values can be used to filter the features. Features that meet this cutoff requirement are considered to have passed the “real feature” test.

### 4. Inference of Whether a Concept is Meaningful

In some embodiments, the extracted clinical features can be further assessed with respect to their clinical relevance, or in other words, whether they are clinically meaningful (e.g., step 204 in FIG. 2). For example, in “Pt with pneumonia and cough”, the feature pneumonia is meaningful but the feature cough is not because it is explained by pneumonia. But, in “Pt with knee pain and cough”, both knee pain and cough may be meaningful. In the former example, cough is a symptom that can explained by the disease, pneumonia. Therefore, cough does not add value to characterization of the clinical condition of the patient.

In one embodiment of a “clinical relevance” test, the system first determines whether a feature is a symptom. This can be done with the assistance of a medical vocabulary, such as SNOMED CT. In some embodiments, this information is included in the clinical knowledge set, which categorizes each feature as disease or finding.

If a feature is a symptom, and is determined to be associated with another feature (a disease) in the list, it can be concluded that the symptom can be explained by the disease. In this scenario, the symptom is considered not clinically relevant (or not clinically meaningful) and thus can be removed.

### 5. Inference of Whether a Concept is Unique

From a group of related features, less granular features may be removed from the list (e.g., step 208 in FIG. 2). Between two related features, such as a parent a child feature, the one that is lower in the hierarchy is considered more granular. For instance, between diabetes mellitus (parent) and diabetes mellitus type 2 (child), diabetes mellitus type 2 is a more granular feature. The hierarchical relationship among features can be determined with knowledge databases such as SNOMED CT. Diabetes mellitus is not unique as it does not add any additional information to the knowledge that the patient has diabetes mellitus type 2.

### 6. Application of Inference for a Specific Data Use

In some embodiments, patients' feature maps are used for research. In such a case, the feature selection process can be adjusted to increase recall or precision. For example, in a study on congestive heart failure (CHF), the feature selection process may be tuned to avoid missing patients with CHF in order to maximize generalizability of the study.

Accordingly, in some embodiments, upon extraction of features from a patient's medical information, an exception is made with features that relate to the subject of a study (e.g., CHF). The exception is used such that features related to the subject can more easily pass the numerous tests discussed herein. For example, if the count of associated features for CHF is 2 and the cutoff value is 3, CHF can still pass.

Likewise, for the “active” test, this exception may also apply so that even patients only have a history of heart attack can be included, if the study so desires. By contrast, the exception is not applied for tests such as the clinical relevance test, which means that symptoms that can be explained by heart attack can still be removed.

Upon extracting the clinical features from a patient's medical information, and evaluating and filtering them according to some or all of the above criteria, a feature map can be generated for the patient (e.g., step 212 in FIG. 2). It would be readily appreciated that not all of the evaluation and filtration techniques are required for every patient. For instance, in one embodiment, the features only need to be evaluated on whether they are real and meaningful, while in another embodiment, the features only need to be evaluated on whether they are active and meaningful, without limitation. The feature map represents a computable phenotype for the patient.

### III. Applications of the Feature Maps

National statistics show only a 67% chance of regular care providers accurately updating the EHR problem list with cancer and a 54% chance with heart failure. Patients receive worse care when the phenotype in their record is incorrect. The patient's direct care is dependent on all providers knowing relevant conditions such as cancer or heart failure. Additionally, indirect care such as disease management and population health resources require the phenotype be accurate. With the feature maps generated with the present technology, the healthcare system would not rely exclusively on the various care providers to manually update EHR lists. Massive data stores already exist in the form of clinical narratives, radiology reports, and pathology reports. These documents, though impossible for a human to read through in minutes, can be readily processed by the machine learning technologies of the instant disclosure to generate feature maps, improving care.

In addition, secondary use of data provides a potentially greater impact in healthcare than primary use. Understanding preferred treatment based on accurate understanding of subgroups and effect of treatment represents a required pillar in precision medicine. Feature maps for a group of patients can be compiled to compare against inclusion and exclusion criteria to obtain a cohort of patients. Subgroups may be tested for exposures and outcomes to understand which therapies work best in which types of patients. In precision medicine, this may result in a data-driven approach to understand preferred therapy for a specific phenotype, such as diabetics with lung cancer. This cohort may be applied value-based healthcare, research, and innovation.

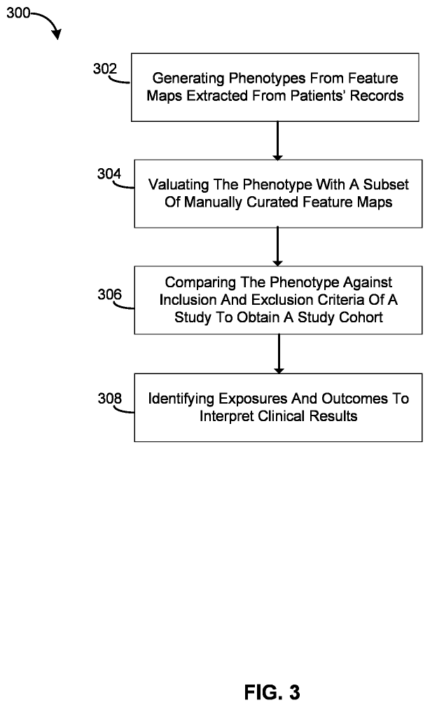

In some embodiments, a method for interpreting clinical results is provided, built upon computer technologies (as illustrated in work flow 300 in FIG. 3). In some embodiments, the method entails generating a phenotype, for each of a plurality of patients, from a feature map extracted from the patient's electronic health record (EHR) (e.g., step 302). In some embodiments, the phenotype is validated with a subset of the feature map that is manually curated (e.g., step 304).

The phenotype can then be compared against inclusion and exclusion criteria of a study to obtain a study cohort (e.g., step 306). As such, exposures and outcomes may be identified within the feature map to interpret clinical results for a cohort of patients.

### IV. Computing Systems for Generating and Using Feature Maps

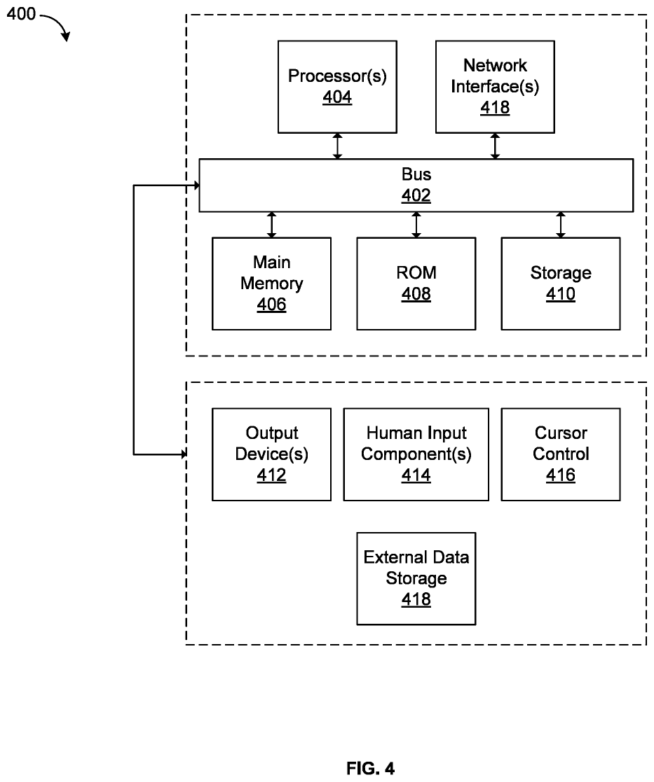

FIG. 4 is a block diagram that illustrates a computer system 400 upon which any embodiments of generation and use of the clinical knowledge set and feature maps, and related technologies may be implemented. The computer system 400 includes a bus 402 or other communication mechanism for communicating information, one or more hardware processors 404 coupled with bus 402 for processing information. Hardware processor(s) 404 may be, for example, one or more general purpose microprocessors.

The computer system 400 also includes a main memory 406, such as a random access memory (RAM), cache and/or other dynamic storage devices, coupled to bus 402 for storing information and instructions to be executed by processor 404. Main memory 406 also may be used for storing temporary variables or other intermediate information during execution of instructions to be executed by processor 404. Such instructions, when stored in storage media accessible to processor 404, render computer system 400 into a special-purpose machine that is customized to perform the operations specified in the instructions.

The computer system 400 further includes a read only memory (ROM) 408 or other static storage device coupled to bus 402 for storing static information and instructions for processor 404. A storage device 410, such as a magnetic disk, optical disk, or USB thumb drive (Flash drive), etc., is provided and coupled to bus 402 for storing information and instructions.

The computer system 400 may be coupled via bus 402 to a display 412, such as a LED or LCD display (or touch screen), for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. In some embodiments, the same direction information and command selections as cursor control may be implemented via receiving touches on a touch screen without a cursor. Additional data may be retrieved from the external data storage 418.

The computer system 400 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and maybe originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage.

The computer system 400 may implement the techniques described herein using customized hard-wired logic, one or more ASICs or FPGAs, firmware and/or program logic which in combination with the computer system causes or programs computer system 400 to be a special-purpose machine. According to one embodiment, the techniques herein are performed by computer system 400 in response to processor(s) 404 executing one or more sequences of one or more instructions contained in main memory 406. Such instructions may be read into main memory 406 from another storage medium, such as storage device 410. Execution of the sequences of instructions contained in main memory 406 causes processor(s) 404 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 410. Volatile media includes dynamic memory, such as main memory 406. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 402. Transmission media can also take the form of acoustic or light waves, such as those generated during radio-wave and infra-red data communications.

Various forms of media may be involved in carrying one or more sequences of one or more instructions to processor 404 for execution. For example, the instructions may initially be carried on a magnetic disk or solid-state drive of a remote computer. The remote computer can load the instructions into its dynamic memory and send the instructions over a telephone line using a component control. A component control local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may retrieve and execute the instructions. The instructions received by main memory 406 may optionally be stored on storage device 410 either before or after execution by processor 404.

The computer system 400 also includes a communication interface 418 coupled to bus 402. Communication interface 418 provides a two-way data communication coupling to one or more network links that are connected to one or more local networks. For example, communication interface 418 may be an integrated services digital network (ISDN) card, cable component control, satellite component control, or a component control to provide a data communication connection to a corresponding type of telephone line. As another example, communication interface 418 may be a local area network (LAN) card to provide a data communication connection to a compatible LAN (or WAN component to communicated with a WAN). Wireless links may also be implemented. In any such implementation, communication interface 418 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world-wide packet data communication network now commonly referred to as the “Internet”. Local network and Internet both use electrical, electromagnetic or optical signals that carry digital data streams. The signals through the various networks and the signals on network link and through communication interface 418, which carry the digital data to and from computer system 400, are example forms of transmission media.

The computer system 400 can send messages and receive data, including program code, through the network(s), network link and communication interface 418. In the Internet example, a server might transmit a requested code for an application program through the Internet, the ISP, the local network and the communication interface 418.

The received code may be executed by processor 404 as it is received, and/or stored in storage device 410, or other non-volatile storage for later execution. Each of the processes, methods, and algorithms described in the preceding sections may be embodied in, and fully or partially automated by, code modules executed by one or more computer systems or computer processors comprising computer hardware. The processes and algorithms may be implemented partially or wholly in application-specific circuitry.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the embodiments should, therefore, be construed in accordance with the appended claims and any equivalents thereof.

The various operations of example methods described herein may be performed, at least partially, by one or more processors that are temporarily configured (e.g., by software) or permanently configured to perform the relevant operations. Similarly, the methods described herein may be at least partially processor-implemented, with a particular processor or processors being an example of hardware. For example, at least some of the operations of a method may be performed by one or more processors. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

The performance of certain of the operations may be distributed among the processors, not only residing within a single machine but deployed across a number of machines. In some example embodiments, the processors may be located in a single geographic location (e.g., within a home environment, an office environment, or a server farm). In other example embodiments, the processors may be distributed across a number of geographic locations.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the subject matter has been described with reference to specific example embodiments, various modifications and changes may be made to these embodiments without departing from the broader scope of embodiments of the present disclosure. Such embodiments of the subject matter may be referred to herein, individually or collectively, by the term “invention” merely for convenience and without intending to voluntarily limit the scope of this application to any single disclosure or concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Although the invention has been described in detail for the purpose of illustration based on what is currently considered to be the most practical and preferred implementations, it is to be understood that such detail is solely for that purpose and that the invention is not limited to the disclosed implementations, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A method for generating a clinical knowledge set, comprising:
identifying, from one or more medical information sources, groups of clinical features that are present together in at least one of the sources;
for each group of features, using a machine learning technique to determine likelihood of relationship; and
generating a clinical knowledge set with the identified groups of related features that meet a minimum threshold of relationship likelihood.

2. The method of claim 1, wherein the medical information sources comprise unstructured data from an electronic health record.

3. The method of claim 1, wherein the medical information sources comprise medical literature.

4. The method of claim 1, wherein the likelihood of relationship is determined at least in part based on a ratio of actual frequency of co-occurrence to the likelihood of the group co-occurring by random chance.

5. The method of claim 1, wherein each group of features is a pair of features.

6. The method of claim 1, wherein the features of at least one of the groups of features have a directional relationship.

7. The method of claim 4, wherein the actual frequency of co-occurrence is determined from narrative notes of electronic health records.

8. The method of claim 4, wherein the actual frequency of co-occurrence is determined from medical literature.

9. The method of claim 1, wherein the likelihood of relationship is determined at least in part based on an industry standard terminology.

10. The method of claim 1, wherein the likelihood of relationship is determined at least in part based on a token distance of the group members and the comparison thereof with the average token distance if the group members were present together by random chance.

11. A method for generating a feature map for a patient, comprising:
extracting, from a patient's medical information, a list of clinical features;
identifying, for each feature in the list, associated features within the list, wherein the association is according to a clinical knowledge set; and
determining features that have a threshold level of associated features within the list, thereby generating a feature map for the patient that includes clinically relevant features.

12. The method of claim 11, wherein the patient's medical information comprises unstructured data from an electronic health record.

13. The method of claim 11, wherein features that are active for the patient are incorporated into the patient map.

14. The method of claim 11, wherein features that are real or relevant to the patient are incorporated into the patient map.

15. The method of claim 11, wherein features that are clinically meaningful are incorporated into the patient map.

16. The method of claim 11, wherein features that are unique are incorporated into the patient map

17. The method of claim 16, wherein between two similar features, the more granular feature is incorporated into the patient map.

18. The method of claim 16, wherein between two similar features, where a disease explains a clinical finding such as a symptom, sign, or exam finding, the disease is incorporated into the patient map but the finding is not.

19. A method for interpreting clinical results, comprising:
generating a phenotype, for each of a plurality of patients, from a feature map extracted from the patient's electronic health record (EHR);
comparing the phenotype against inclusion and exclusion criteria of a study to obtain a study cohort; and
identifying exposures and outcomes within the feature map to interpret clinical results for a cohort of patients.

20. The method of claim 19, further comprising validating the phenotype with a subset of the feature map that is manually curated.
